(19) United States
(12) Patent Application Publication
(10) Pub. No.: US 2015/0088253 A1
(43) Pub. Date: Mar. 26, 2015

(54) Systems and Methods for Power Management of Implantable Ophthalmic Devices

(71) Applicant: Elenza, Inc., Roanke, VA (US)

(72) Inventors: Walter Doll, Gasel (CH); Jean-Noel Fehr, Neuchatel (CH); Urban Schnell, Münchenbuchsee (CH); Roelof Trip, Suwanee, GA (US); Amitava Gupta, Roanke, VA (US)

(21) Appl. No.: 14/390,680

(22) PCT Filed: Mar. 14, 2013
(86) PCT No.: PCT/US2013/031533

§ 371 Date: Oct. 3, 2014

Related U.S. Application Data
(60) Provisional application No. 61/621,193, filed on Apr. 6, 2012.
(60) Provisional application No. 61/637,564, filed on Apr. 24, 2012.
(60) Provisional application No. 61/638,016, filed on Apr. 25, 2012.

(51) Int. Cl. A61F 2/16; G01R 31/36; H02J 7/00

(52) U.S. Cl. CPC A61F 2/1624; H02J 7/0052; H02J 7/007; G01R 31/362; A61F 2002/1697
USPC 623/6.22; 320/160

(57) ABSTRACT

Systems and methods of the disclosure relate to managing power consumption of an implantable device, such as an implantable ophthalmic device, that includes one or more rechargeable batteries and a processor operably coupled to the rechargeable batteries. The processor can be configured to implement a quick-charge process that includes charging each rechargeable battery for a first time interval using a first constant current, for a second time interval using a second constant current less than the first constant current, and for a third time interval using a constant voltage. This quick charge process is faster than conventional charging. The processor also manages discharge of two batteries in an alternating fashion so as to increase time between charging cycles, reduce the total number of charging cycles, and extend battery life.

### CROSS-REFERENCE TO RELATED PATENT APPLICATION

This application claims the benefit of: U.S. Provisional Application No. 61/621,193 filed Apr. 6, 2012 and titled “An Application Specific Integrated Circuit (ASIC) For Use In Intraocular Implants”; U.S. Provisional Application No. 61/637,564 filed Apr. 24, 2012 and titled “Electronic Control System for an Intraocular Implant”; and U.S. Provisional Application No. 61/638,016 filed Apr. 25, 2012 and titled “Rechargeable Batteries for Intraocular Implants.” Each of the above-referenced applications is incorporated herein by reference in its entirety.

### BACKGROUND

There are two major conditions that affect an individual's ability to focus on near and intermediate distance objects: presbyopia and pseudophakia. Presbyopia is the loss of accommodation of the crystalline lens of the human eye that often accompanies aging. In a presbyopic individual, this loss of accommodation first results in an inability to focus on near distance objects and later results in an inability to focus on intermediate distance objects. It is estimated that there are approximately 90 million to 100 million presbyopes in the United States. Worldwide, it is estimated that there are approximately 1.6 billion presbyopes.

The standard tools for correcting presbyopia are reading glasses, multifocal ophthalmic lenses, and contact lenses fit to provide monovision. Reading glasses have a single optical power for correcting near distance focusing problems. A multifocal lens is a lens that has more than one focal length (i.e., optical power) for correcting focusing problems across a range of distances. Multifocal optics are used in eyeglasses, contact lenses, and intra-ocular lenses (IOLs). Multifocal ophthalmic lenses work by means of a division of the lens's area into regions of different optical powers. Multifocal lenses may be comprised of continuous surfaces that create continuous optical power as in a Progressive Addition Lens (PAL). Alternatively, multifocal lenses may be comprised of discontinuous surfaces that create discontinuous optical power as in bifocals or trifocals. Contact lenses fit to provide monovision are two contact lenses having different optical powers. One contact lens is for correcting mostly far distance focusing problems and the other contact lens is for correcting mostly near distance focusing problems.

Pseudophakia is the replacement of the crystalline lens of the eye with an IOL, usually following surgical removal of the crystalline lens during cataract surgery. For all practical purposes, an individual will get cataracts if he or she lives long enough. Furthermore, most individuals with cataracts will have a cataract operation at some point in their lives. It is estimated that approximately 1.2 million cataract surgeries are performed annually in the United States. In a pseudophakic individual, the absence of the crystalline lens causes a complete loss of accommodation that results in an inability to focus on either near or intermediate distance objects.

Conventional IOLs are monofocal, spherical lenses that provide focused retinal images for far objects (e.g., objects over two meters away). Generally, the focal length (or optical power) of a spherical IOL is chosen based on viewing a far object that subtends a small angle (e.g., about seven degrees) at the fovea. Unfortunately, because monofocal IOLs have a fixed focal length, they are not capable of mimicking or replacing the eye's natural accommodation response. Fortunately, ophthalmic devices with electro-active elements, such as liquid crystal cells, can be used to provide variable optical power as a substitute for the accommodation of an damaged or removed crystalline lens. For example, electro-active elements can be used as shutters that provide dynamically variable optical power as disclosed in U.S. Pat. No. 7,926,940 to Blum et al., which is incorporated herein by reference in its entirety.

### SUMMARY

Embodiments of the disclosed technology include an implantable device, such as an implantable ophthalmic device suitable for treating aphakia or pseudophakia. The device can include a first rechargeable battery and a processor operably coupled to the first rechargeable battery. The processor can be configured to charge the first rechargeable battery for a first time interval using a first constant current. The processor can also be configured to charge the first rechargeable battery for a second time interval using a second constant current less than the first constant current. The processor can also be configured to charge the first rechargeable battery for a third time interval using a constant voltage.

In some implementations, the first rechargeable battery is a solid-state lithium battery or a lithium-ion battery. In some implementations, the first rechargeable battery has a volume of less than five cubic millimeters. In some implementations, the processor is configured to determine an end of the first time interval when a voltage of the first rechargeable battery exceeds a first threshold voltage. In some implementations, the processor is configured to determine an end of the second time interval when the voltage of the first rechargeable battery exceeds a second threshold voltage. In some implementations, the second constant current is substantially equal to half the first constant current. For example, the first constant current can be from about 20 to about 40 μA.

In some implementations, the processor can also include a power conversion module. The power conversion module can be configured to receive power from a power source external to the implantable device and convert the power to the first constant current, the second constant current, and the constant voltage. For example, the power source can be a radio-frequency source or a light source.

In some implementations, the device can include a second rechargeable battery operably coupled to the processor. The processor can be configured to charge the second rechargeable battery for a fourth time interval using a third constant current. The processor can be configured to charge the second rechargeable battery for a fifth time interval using a fourth constant current less than the third constant current. The processor can also be configured to charge the second rechargeable battery for a sixth time interval using a second constant voltage. In some implementations, the device can also include an electro-active element operably coupled to the processor. The electro-active element can be configured to modulate at least one optical characteristic of the implantable device.

Another aspect of the disclosed technology relates to a method of charging a battery. The method can include charging the rechargeable battery for a first time interval using a first constant current. The method can include determining that a voltage of the rechargeable battery exceeds a first threshold value. The method can include charging the rechargeable battery for a second time interval using a second constant current less than the first constant current. The method can include determining that the voltage of the rechargeable battery exceeds a second threshold value. The method can also include charging the rechargeable battery for a third time interval using a constant voltage.

Another aspect of the disclose technology relates to an intraocular optic. The optic can include an electro-active element configured to vary an optical characteristic. The optic can include a sensor configured to generate a sensor signal within less than about 100 milliseconds in response to sensing a change in light level or a physiological response. The optic can include a first control circuit, operably coupled to the sensor, configured to sample the sensor signal and to generate an actuation signal within 100 milliseconds of sampling the sensor signal in response to the sensor signal. The optic can also include a second control circuit operably coupled to the first control circuit and to the electro-active element. The second control circuit can be configured to receive the actuation signal. The second control circuit can be configured to transition from a low-power state to a high-power state and actuate the electro-active element within about 5 milliseconds of receiving the actuation signal so as to vary the optical characteristic of the intraocular optic in response to the actuation signal. The second control circuit can also be configured to transition from the high-power state to the low-power state within about 5 milliseconds of actuating the electro-active element so as to minimize current leakage from the second control circuit.

In some implementations, the first control circuit is configured to sample the sensor signal at a period of about 200 milliseconds to about 310 milliseconds. In some other implementations, the first control circuit is configured to sample the sensor signal aperiodically.

Another aspect of the disclosed technology relates to a method of altering an optical characteristic of an intraocular optic in response to a change in light level or a physiological response. The method can include sensing the change in light level or the physiological response. The method can include generating a sensor signal within about 100 milliseconds of sensing the change in light level or a physiological response. The method can include sampling the sensor signal with a first control circuit. The method can include generating an actuation signal, with the first control circuit, within 100 milliseconds of sampling the sensor signal. The method can include actuating the intraocular optic based on the actuation signal so as to minimize current leakage from the second control circuit. The method can include receiving the actuation signal at a second control circuit. The method can include transitioning the second control circuit from a low-power state to a high-power state in response to the actuation signal. The method can include actuating an electro-active element with the second control circuit so as alter the characteristic of the intraocular optic within about 5 milliseconds of receiving the actuation signal The method can also include transitioning the second control circuit from the high-power state to the low-power state within about 5 milliseconds of actuating the electro-active element so as to minimize current leakage from the second control circuit.

Another aspect of the disclosed technology relates to an implantable device. The implantable device can include a first rechargeable battery having a first voltage, a second rechargeable battery having a second voltage, and a processor operably coupled to the first rechargeable battery and the second rechargeable battery. The processor can be configured to determine that the first voltage has fallen below the second voltage. The processor can be configured to select the second rechargeable battery to discharge in response to the determination that the first voltage has fallen below the second voltage. The processor can be configured to determine that the second voltage has fallen below the first voltage. The processor can be configured to select the first rechargeable battery to discharge in response to the determination that the first voltage has fallen below the second voltage. In some implementations, the processor is configured to perform these steps iteratively.

In some implementations, the first rechargeable battery or the second rechargeable battery includes at least one of a solid-state lithium battery and a lithium-ion battery. The first rechargeable battery or the second rechargeable battery can have a volume of less than five cubic millimeters. In some implementations, the processor is further configured to determine that the first voltage has fallen below a first threshold, determine that the second voltage had fallen below a second threshold, and cause a reduction in power flow from the first rechargeable battery and the second rechargeable battery in response to the determination that the first voltage has fallen below the first threshold and the determination that the second voltage had fallen below the second threshold.

In some implementations, the device also includes an electro-active element operably coupled to the processor, the first rechargeable battery, and the second rechargeable battery. The electro-active element can be configured to vary an optical characteristic of the implantable device when powered by at least one of the first rechargeable battery and the second rechargeable battery.

Another aspect of the disclosed technology relates to an intraocular optic. The intraocular optic includes a sensor configured to sense at least one of a light level and a physiological response. The intraocular optic also includes an electro-active element to vary at least one optical characteristic of the intraocular implant. The intraocular optic also includes a first control circuit, operably coupled to the sensor, configured to sample the sensor signal and to generate an actuation signal within 100 milliseconds of sampling the sensor signal in response to the sensor signal. The intraocular optic also includes a second control circuit, operably coupled to the first control circuit and to the electro-active element.

The second control circuit can be configured to receive the actuation signal. The second control circuit can be configured to transition from a low-power state to a high-power state and actuate the electro-active element so as to vary the at least one optical characteristic of the intraocular optic in response to the actuation signal. The second control circuit can be configured to transition from the high-power state to the low-power state of actuating the electro-active element so as to minimize current leakage from the second control circuit.

The intraocular optic can also include at least one rechargeable battery, operably coupled to the first control circuit and the second control circuit. The at least one rechargeable battery can be configured to provide power to the second control circuit when the second control circuit is in the high-power state and to be recharged by a first constant current provided by the first control circuit over a first time interval, a second constant current less than the first constant current provided by the first control circuit over a second time interval after the first time interval, and a constant voltage provided by the first control circuit over a third time interval after the second time interval.

In some implementations, the at least one rechargeable battery includes a first rechargeable battery having a first voltage and a second rechargeable battery having a second voltage. The first control circuit can further be configured to provide power to the second control circuit by determining that the first voltage has fallen below the second voltage, selecting the second rechargeable battery to discharge in response to the determination that the first voltage has fallen below the second voltage, determining that the second voltage has fallen below the first voltage, and selecting the first rechargeable battery to discharge in response to the determination that the first voltage has fallen below the second voltage. In some implementations, the first control circuit can be configured to iterate these steps.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

The accompanying drawings, which are incorporated in and constitute a part of this specification, illustrate embodiments of the disclosed technology and together with the description serve to explain principles of the disclosed technology.

FIG. 1A is a perspective view of an intraocular lens (IOL), according to an illustrative implementation.

FIG. 1B is an exploded view of the IOL shown in FIG. 1A, according to an illustrative implementation.

FIG. 2 illustrates first and second ASICs suitable for use in an implantable device such as the IOL of FIG. 1A, according to an illustrative implementation.

FIG. 3 is a state transition diagram for the first and second ASICs shown in FIG. 2, according to an illustrative implementation.

FIG. 4 is a timing diagram showing the signal processing characteristics of an implantable device such as the IOL of FIG. 1A, according to an illustrative implementation.

FIG. 5A is diagram of a lithium ion rechargeable battery suitable for use in an implantable device such as the IOL of FIG. 1A, according to an illustrative implementation.

FIG. 5B is a diagram of a rechargeable solid-state battery suitable for use in an implantable device such as the IOL of FIG. 1A, according to an illustrative implementation.

FIG. 6 is circuit diagram showing a battery charging circuit suitable for use in an implantable device such as the IOL of FIG. 1A, according to an illustrative implementation.

FIG. 7 is a graph showing the charging characteristics of a battery such as the lithium ion battery of FIG. 5A or the solid-state battery of FIG. 5B, according to an illustrative implementation.

FIG. 8 is a flow diagram of a process for charging a rechargeable battery, according to an illustrative implementation.

FIG. 9 is a graph showing the discharging characteristics of the two batteries shown in FIG. 2, according to an illustrative implementation.

FIG. 10 is a flow diagram of a process for discharging two rechargeable batteries substantially simultaneously, according to an illustrative implementation.

### DETAILED DESCRIPTION

Presently preferred embodiments of the invention are illustrated in the drawings. An effort has been made to use the same or like reference numbers to refer to the same or like parts.

Electronic Control Systems for Implantable Ophthalmic Devices

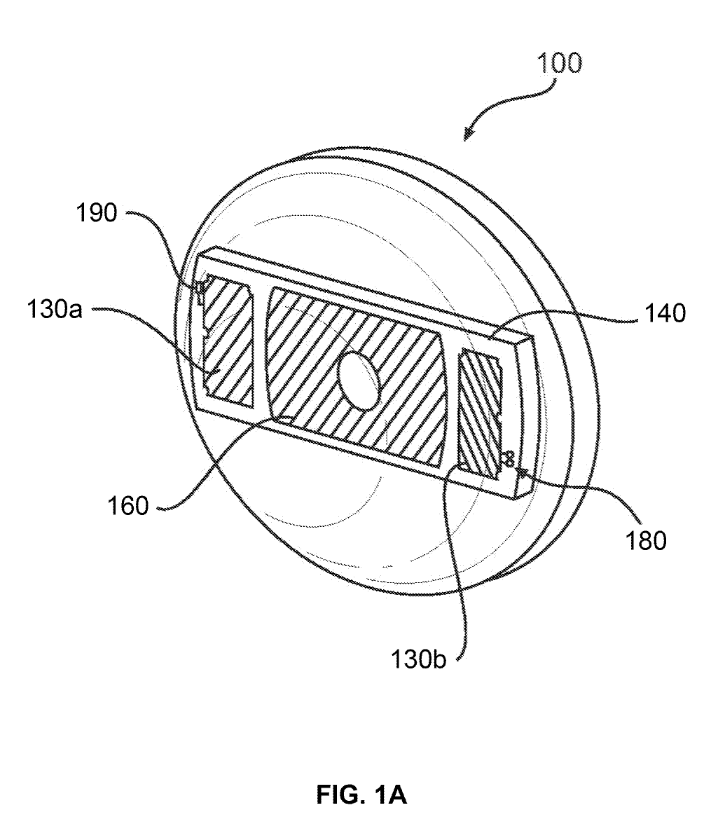

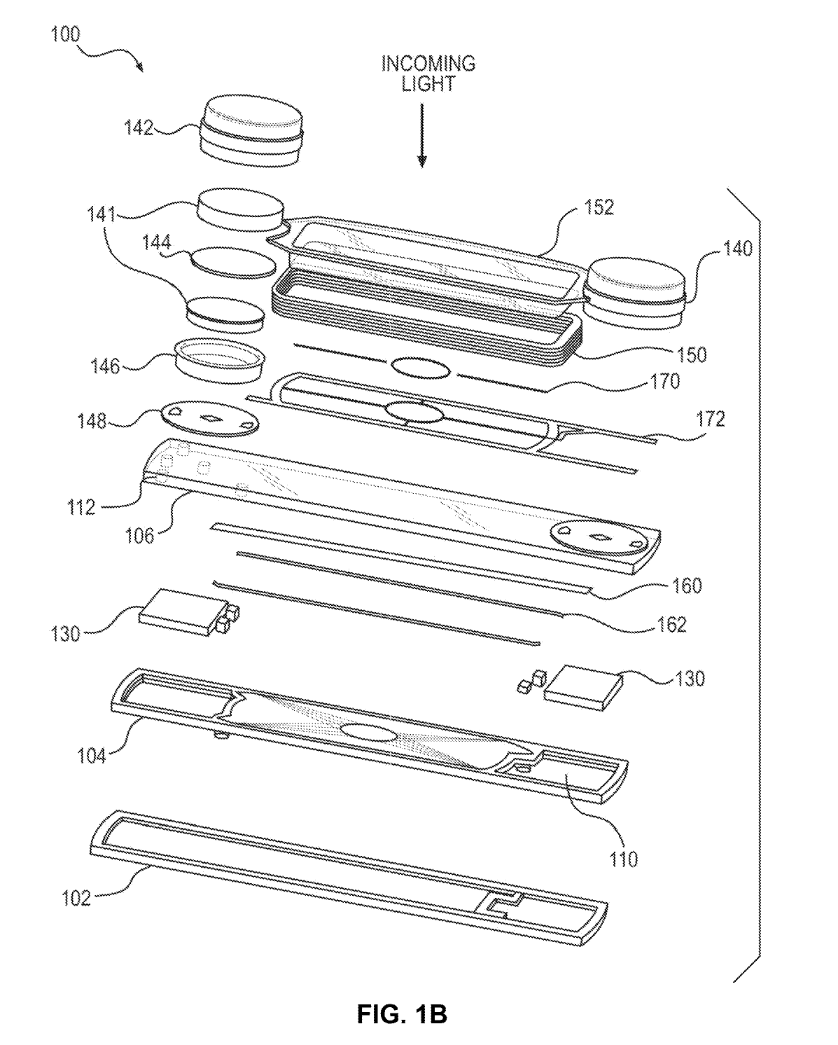

This invention generally relates to power management of implantable devices, such as implantable ophthalmic devices. FIG. 1A shows an exemplary implantable ophthalmic device 100, such as an intraocular lens (IOL), for use in dynamically correcting or adjusting a patient's vision. The device 100 includes a power supply—in this case, a rechargeable battery 140—coupled to a first application-specific integrated circuit (ASIC) 130a and a second ASIC 130b. The battery 140 provides current at a relatively low voltage, e.g., about 4 V or less, to both ASICs 130a and 130b. The first ASIC 130a is coupled to an electro-active element 160 that operates at a relatively high voltage, e.g., about 5 V to about 11 V. And the second ASIC 130b operates at a lower voltage (e.g., about 5 V or less) to monitor environmental and/or physiological conditions for indications of accommodative triggers and to control the first ASIC 130a.

The electro-active element 160 provides a dynamically variable optical power and/or depth of field that adds to the (optional) static optical power provided by the device's curved surface. For example, the electro-active element 160 can act as a variable diameter aperture that opens and closes in response to accommodative triggers to increase or decrease the depth of field. The device 100 may also include a sensor 180, such as a photodetector or ion sensor, for detecting the eye's accommodative response and an antenna 190 for receiving radio-frequency power or data communication. The electronics can be embedded or otherwise hermetically sealed inside the device 100 itself, which may be molded of glass, resin, plastic, or any other suitable material.

FIG. 1B is an exploded view of the implantable ophthalmic device 100 shown in FIG. 1A. The device includes cavities 110 and feedthroughs 112 that are hermetically sealed to prevent leakage of foreign material from the device 100 into the eye. As defined herein, a hermetically sealed cavity or feedthrough is a cavity or feedthrough that passes an American Society for Testing and Materials (ASTM) E493/E493M-11 helium leak test with a leak rate of less than 5.0×10⁻¹² Pa m³s⁻¹. In some embodiments, the amount of helium that leaks through a hermetic seal during a helium leak is undetectable, i.e., it is lower than the normal atmospheric concentration of helium.

The assembly 100 includes electronic components—in this case, ASICs 130 that have different functional blocks and may be populated with additional electronic components—disposed within the cavities 110 in an intermediate wafer 104. The ASICs 130 can be populated with subcomponents using thermo-compression bonding via TiAgNiAu pads material with mechanical tolerances of ±10 μm in all three dimensions. The assembly may also include AgPb capacitors (not shown), such as 01005 SMD surface-mount capacitors, that are bonded to a printed circuit board (PCB) (not shown) with anisotropic conductive adhesives with a lateral alignment tolerance of ±50 μm. In preferred embodiments, the total height from the surface of the PCB to the top of the capacitor is about 255±10 μm.

The cavities 110 are defined by sealing apertures in the intermediate wafer 104 between a bottom wafer 102 and a top wafer 106, which can be bonded together using laser fusion bonding, pressure bonding, and/or anodic bonding. Other elements, such as the electro-active cell 160 and an obscuration 162, which comprises an opaque layer that absorbs more than 90% of incident light, may be affixed to or sealed between the wafers 102, 104, and 106, which can be made of borosilicate glass (e.g., Borofloat® 33 or D263™), pure silica (SiO₂), fused silica, or any other suitable material.

The ASICs 130 are electrically connected to batteries 140 via the feedthroughs 112 that run through the top wafer 106. The batteries 140, which may be rechargeable, include cells 141 held apart by a separator 144 and covered in a casing 142 that provides leakage protection for up to 25 years or more. A battery casing isolation ring 146 insulates the cells 141 from the rest of the assembly 100, and a battery insert plate 148 hold the battery 140 and its components in place with respect to the top wafer 106.

The assembly 100 also includes an inductive antenna coil 150 and a photovoltaic cell 170 that can be used to recharge the batteries 140. The coil 150 and the photovoltaic cell 170 can also be used for wireless communication with external processors, e.g., to update and/or extract information store in memory on one or both of the ASICs 130. The photovoltaic cell 170 can also be used to detect accommodative triggers, changes in pupil diameter, and/or other physiological or environmental indications with an average sensitivity of about 0.48 nA/lux mm2. In some embodiments, the assembly 100 includes two TiAu—PIN—ZnO photovoltaic cells: a first cell with diameter of about 1.175-1.225 mm and a second cell with dimensions of about 0.1 mm×1.8 mm. In some examples, the coil 150 has about fifteen windings arranged about a perimeter of 5.7 mm×2.6 mm.

The coil 150 and photovoltaic cell 170 are also be in electrical communication with the ASICs 130 via the feedthroughs 112. For instance, a battery charger (not shown) in one of the ASICs 130 may control the recharging process as described in PCT/US2011/040896 to Fehr et al., which is incorporated herein by reference in its entirety. Similarly, a processor in one of the ASICs 130 may receive signals from the photovoltaic cell 170 representing the pupil diameter as also described in PCT/US2011/040896 to Fehr et al. The processor may also control the diameter of an aperture defined by the electro-active cell 160 in response to signals from the photovoltaic cell 170, e.g., as described in U.S. Pat. No. 7,926,940 to Blum et al., which is also incorporated herein by reference in its entirety.

The implantable ophthalmic device 100 shown in FIG. 1B is illustrative only. In some implementations, the implantable ophthalmic device 100 may include more or fewer components than are shown in FIG. 1B. The arrangements of the components can also be different in various implementations. For example, the coil 150 can be wrapped around the batteries 140. Winding the coil 150 around The batteries 140 provide good mechanical stability for the coil 150, but may impose constraints on how the implant is assembled (e.g., batteries 140 before the coil 150). The batteries 140 may also interfere with inductive coupling between the coil 150 and external electromagnetic sources (antennas).

The coil 150 can also be wound around a separate support 152. In some cases, an optic, such as an aspheric lens or a spherical lens, may be integrated into the support 152. For example, a portion of the support's outer surface may be curved or patterned to refract or diffract incident light. Using a separate support 152 also increases the flexibility of the manufacturing process by obviating any need to install certain components (e.g., batteries 140) before the coil 150. It also makes it possible to optimize the coil's coupling efficiency by allowing the coil 150 to follow a path away from potential sources of interference. However, using the separate support 152 may increase the manufacturing complexity and total mass of the implantable ophthalmic device.

Alternatively, the coil 150 may be self-sustaining, i.e., it may not require any additional support. Like other coils, self-sustaining coils should be positioned within acceptable mechanical tolerances, and may be held in place with respect to the wafers using an adhesive. Care should be taken to prevent self-sustaining coils from deforming during encapsulation of the electronics assembly 100 in acrylic, resin, or other media.

The coil 150 can also be sealed within a cavity to eliminate the need for feedthroughs between the coil 150 and the ASICs 130. In this example, the coil 150 is embedded inside a 0.3 mm thick glass “disc” with two electrical connection on one side of the “disc”. Because the coil 150 is hermetically sealed within the cavity, non-biocompatible material can be used for the coil wires (e.g., copper instead of gold) and for the insulation layer. Sealing the coil 150 within a cavity also eliminates the need to use biocompatible conductive materials to connect the coil 150 to components within the cavity.

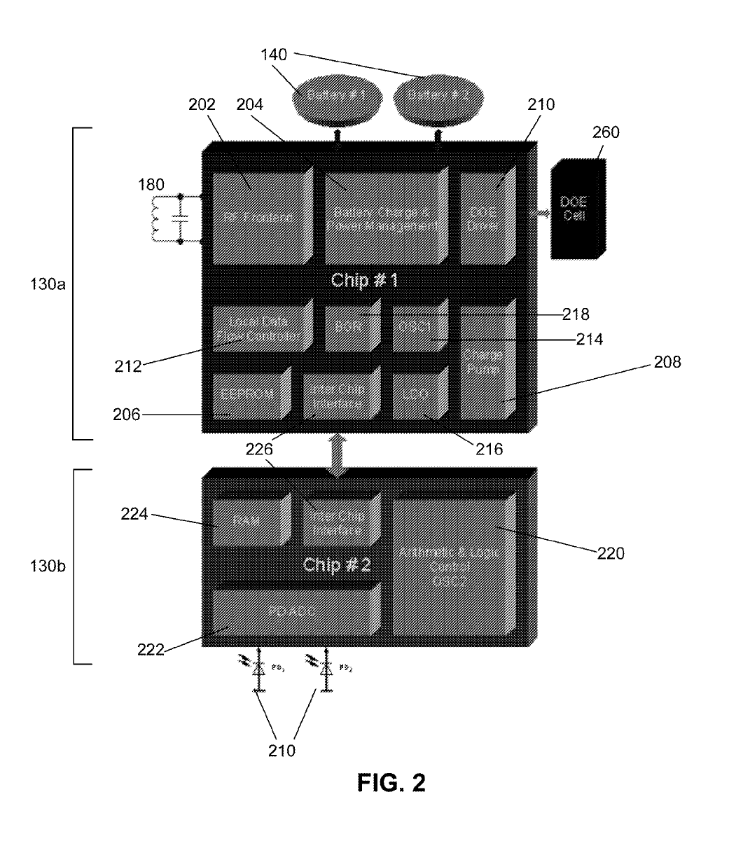

FIG. 2 illustrates the first and second ASICs 130a and 130b of the IOL 100 of FIG. 1A, according to an illustrative implementation. The first ASIC 130a includes a radio-frequency (RF) frontend 202 with a magnetic antenna 180 for power and data management. In some implementations, the magnetic antenna 180 can receive power from an external radio-frequency source, which can be used by a battery charge and power management module 204 to charge the batteries 140. The battery charge and power management module 204 also discharges the batteries 140 to power the first and second ASICs 130a and 130b as described in greater detail below.

The battery charge and power management module 204 is also coupled to a diffractive optical element (DOE) driver 210 in the first ASIC 130a that actuates a diffractive optical element (DOE) 260, which may correspond to the electroactive element 160 of FIG. 1A. As explained below, the first ASIC 130a remains in a low-power state (also known as a sleep or inactive state) unless the DOE 260 is being actuated, in which case the first ASIC 130a enters a high-power or active state. When in the active state, the first ASIC 130a activates a charge pump 208 coupled to the battery charge and power management module 204 generates a high voltage signal for actuating the DOE 260 (e.g., opening or closing an aperture defined by the DOE 260). Once actuation is complete, the first ASIC 130a returns to the low-power state to reduce leakage current from the charge pump 208 or the battery charge and power management module 204.

The first ASIC 130a also includes an electrically erasable programmable read-only memory (EEPROM) module 206 for storing system parameters. The first ASIC 130a can also include a local data flow controller module 212 that can be configured to control data transmission between the various components of the first ASIC 130a. An oscillator 214 in the first ASIC 130a provides a timing signal to synchronize communication between the components of the both the first ASIC 130a and the second ASIC 130a.

In some implementations, the first ASIC 130a can also include a low-dropout (LDO) regulator 216 and a bandgap reference (BGR) circuit 218. The LDO regulator 216 is a DC linear voltage regulator that converts unregulated battery voltage into a regulated power supply voltage. The BGR circuit 218 is a voltage reference circuit that emits a reference voltage (e.g., 1.25 V) that is does not vary much, if at all, with temperature. In other words, the BGR circuit's reference voltage remains stable despite changes in temperature. The reference voltage from the BGR circuit 218 is input into other ASIC blocks, including the power management block 204, which comprises a comparator (not shown) that compares the battery voltage to the reference voltage to determine when the batteries 140 should charged, discharged, etc., as described in greater detail below.

The second ASIC 130b is coupled to one or more photodetectors 210. The photodetectors 210 can determine an ambient light level in the environment surrounding the eye. The ambient light level determined by the photodetectors 210 can be converted to a digital signal by an analog to digital converter 222. The resulting digital signals can be used by the second ASIC 130b to control the operation of the first ASIC 130a. For example, the second ASIC 130b can include a logic module 220 for implementing an actuation algorithm based on the ambient light level. The results of the algorithm can then be communicated to the first ASIC 130a, which can actuate the DOE accordingly. The second ASIC 130b can also include a random access memory (RAM) module 224, which can be configured to store information such as ambient light levels determined by the photodetectors 210, digital outputs from the ADC module 222, and parameters to be used by the logic module 220. The first ASIC 130a and the second ASIC 130b can each include a respective inter-chip interface module 226 to facilitate communication between the first ASIC 130a and the second ASIC 130b.

Operation of an Implantable Ophthalmic Device

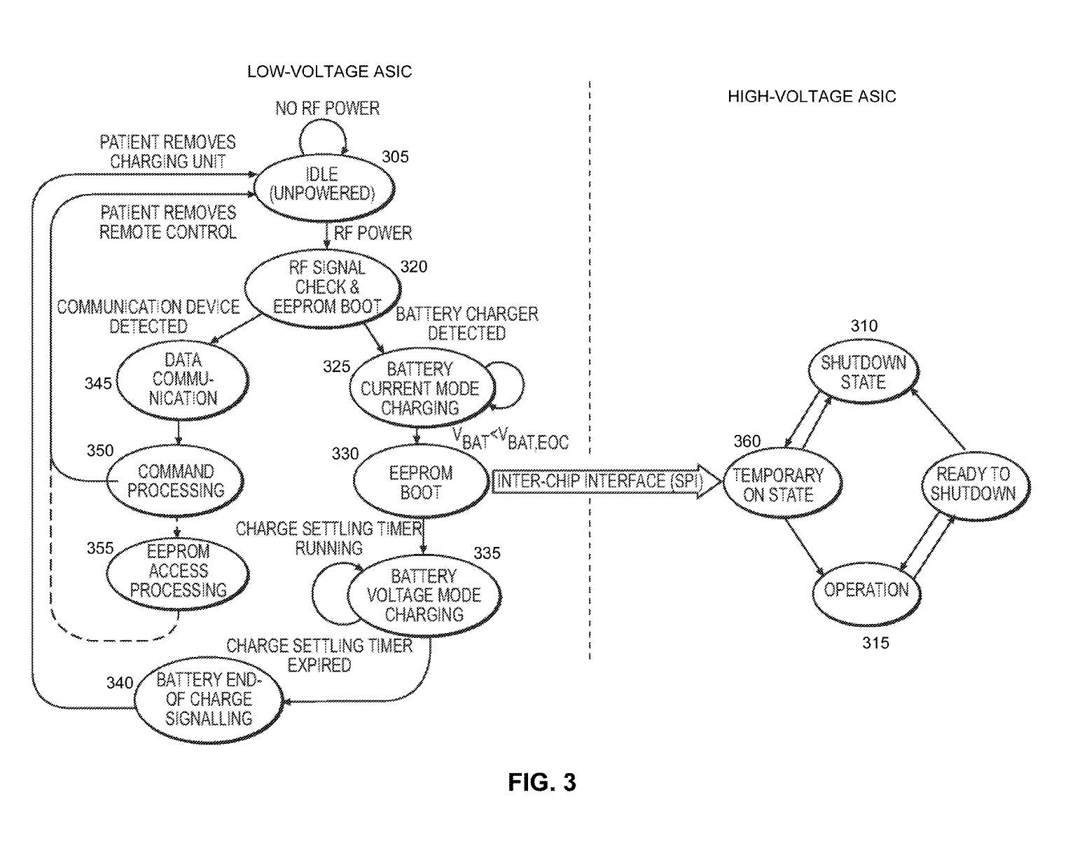

FIG. 3 is a state transition diagram for the ASICs shown in FIG. 2. The first and second ASICs can have four main power conditions corresponding to different device states, all of which are listed below in TABLE 1. When the system is off, the low-voltage ASIC (e.g., second ASIC 130b) is in an unpowered idle mode 305, and the high-voltage ASIC (e.g., first ASIC 130a) is in a sleep (shutdown) state 310. Under normal operating conditions, e.g., when the user is going about his or her day, the system operates in autonomous therapeutic function mode to provided automatic accommodation upon detection of accommodative responses. The high-voltage ASIC switches to its operational mode 315 and the low-voltage ASIC remains in idle mode when the device is operating in autonomous therapeutic function mode. The device can also be charged and/or communicate wirelessly with external readers while continuing to provide autonomous therapeutic function for the patient. When charging and providing autonomous therapeutic function, the low-voltage ASIC switches to an externally (e.g., inductively) powered state and the high-voltage ASIC remains in its operational mode. The device may also be charged and/or communicate wirelessly without providing autonomous therapeutic function, in which case the high-voltage ASIC shuts down to reduce power consumption and current leakage.

In each case, the low-voltage ASIC can change the state of the high-voltage ASIC by issuing an “interrupt” signal (spi_vdd) to the high-voltage ASIC via an interchip data interface. If high-voltage ASIC is in a power-down state 310, the low-voltage ASIC initiates a power-on of the high-voltage ASIC, setting it to a temporary on state 360, and sets the interchip data interface into a command receive state.

TABLE 1
ASIC Powering Conditions

| | High-Voltage | |
|---|---|---|
| Low-Voltage ASIC | ASIC | Device State |
| IDLE (unpowered) | Shutdown | System Off |
| IDLE (unpowered) | Operation | Autonomous Therapeutic Function |
| RF powered | Operation | Charging or communication in |
| (blank states in | | progress, therapeutic function |
| FIG. 3) | | running |
| RF powered | Shutdown | Charging or communication in |
| (blank states in | | progress, therapeutic function |
| FIG. 3) | | disabled |

As shown in FIG. 3, the low-voltage ASIC may transition from idle state 305 to an operational state 320 through application of an RF carrier signal to an RF front-end resonant circuit in the ophthalmic device. For example, the patient may use a remote control to actuate or upload new data to the ophthalmic device. Alternatively, the patient may charge the ophthalmic device with a charging unit.

When the RF front-end resonant circuit detects an rf carrier signal, it sends a signal to a control logic section block on the low-voltage ASIC. At the beginning of the application of an RF field, the control logic section block may be unaware of whether the RF field is being applied for communication and/or battery charging, or both. The logic section block checks the RF signal to determine whether to enter communication mode or battery charging mode. At the same time, a local memory (EEPROM) boot sequence is initiated to transfer the relevant control bits required on the low-voltage ASIC to local data latches. These bits may include trim bits for the rf tuning or control bits for battery charging.

If the logic section block determines that it should enter communication mode, it either begins data communication with the remote control (state 345), processes commands from the remote control (state 350), and stores/retrieves information from local memory (state 355). If the logic section block determines that it should enter charging mode, it begins constant current charging (state 325), then boots the EEPROM (state 330) and switches to constant voltage charging once the battery reaches a predetermined charge level as described above (state 335). Once communication or charging is finished, the patient removes the remote control or the charging unit, and the low-voltage ASIC returns to its idle state 305.

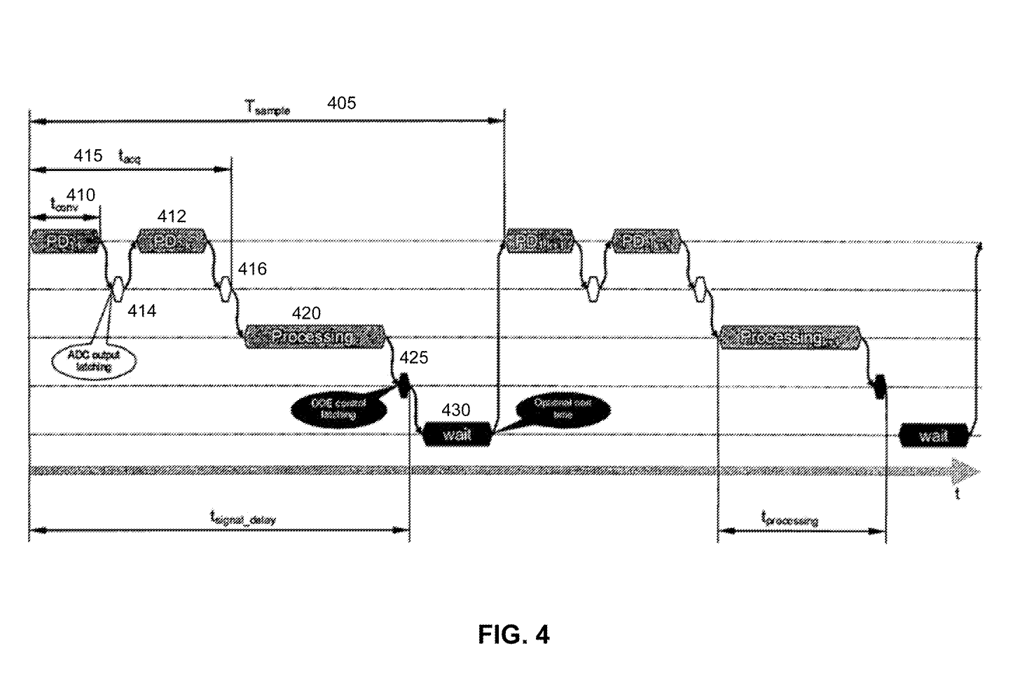

FIG. 4 is a timing diagram showing the signal processing characteristics of an implantable ophthalmic device such as the one shown in FIG. 1A. The timing diagram shows two cycles of a periodic process that can be implemented by the IOL. Each cycle lasts for a sample period 405 denoted by Tₛₐₘₚₗₑ. In some implementations, Tₛₐₘₚₗₑ is in the range of about 200 ms to about 310 ms. This sample period duration enables the implantable ophthalmic device to detect and respond to accommodative triggers quickly enough to mimic accommodation in a healthy eye.

Each cycle begins with a pair of sequential photodetector polling periods 410 and 412, each of which is about 0 ms to about 40 ms (e.g., 5 ms, 10 ms, 20 ms, 30 ms, or any other value less than 40 ms). During the first polling period 410, control logic in the low-voltage ASIC polls, integrates, or samples an analog electrical signal, such as a photocurrent, charge packet, or change in voltage, from a first photodetector. An ADC in the low-voltage ASIC converts this analog signal into a digital signal representative of a light level detected by the first photodetector, and the digital signal is latched during a first ADC latching period 414. The electrical signal output by the first photodetector is then converted to a digital signal using an analog-to-digital converter (e.g., ADC 220 in FIG. 2). A second photodetector converts ambient light to an analog signal during the second polling period 412, which is then converted to a digital signal and latched by the ADC during a second latching period 414. The polling and latching periods occur during an acquisition period 415 that lasts for a time t_{acq}, which may be less than about 100 ms (e.g., 10 ms, 20 ms, 30 ms, 40 ms, 50 ms, 60 ms, 70 ms, 80 ms, 90 ms, or any other value between 0 and 100 ms).

The logic module in the low-voltage ASIC processes the digital signals during a processing period 420 that begins after the second latching period 414. During processing, the logic module may compare the digital signals to values stored in a look-up table in the memory. If the comparison indicates that the ambient light levels have changed in a way indicative of the presence of an accommodative trigger, the low-voltage ASIC generates an actuation signal, which can be used to control an electroactive element such as the DOE 260 of FIG. 2. In some implementations, the processing period 420 lasts less than about 100 ms (e.g., 10 ms, 20 ms, 30 ms, 40 ms, 50 ms, 60 ms, 70 ms, 80 ms, 90 ms, or any other value between 0 and 100 ms). The actuation signal is transmitted to the high-voltage ASIC (e.g., first ASIC 130a in FIG. 2), which responds by transitioning from a sleep state to an active state, actuating the DOE 260, and returning to the sleep state during a control latching period 425 of about 5 ms or less (e.g., about 1 ms, 2 ms, 3 ms, or 4 ms). Because the high-voltage ASIC is only briefly in an active state (e.g., for a duty cycle of under about 3% given the total cycle period 405), the high-voltage ASIC consumes less power and has a lower leakage current. After an optional wait period 430 following DOE 260 actuation, the cycle can begins again.

The exact length of the control latching period 425 depends at least in part on the degree and direction of actuation experience by the DOE 260. For instance, it may take the DOE 260 more time to transition from a fully transmissive state (e.g., 90% transmissive) to a fully opaque state (e.g., 0% transmissive) than from a partially transmissive state (e.g., 60% transmissive) to a partially opaque state (e.g., 10% transmissive). Similarly, the DOE 260 may exhibit hysteresis: for instance, it may take longer to transition from an opaque state to a transmissive state than vice versa. The number, arrangement, and location of the actuated pixels in the DOE 260 may also affect the length of the control latching period 425.

Rechargeable Batteries for Implantable Ophthalmic Devices

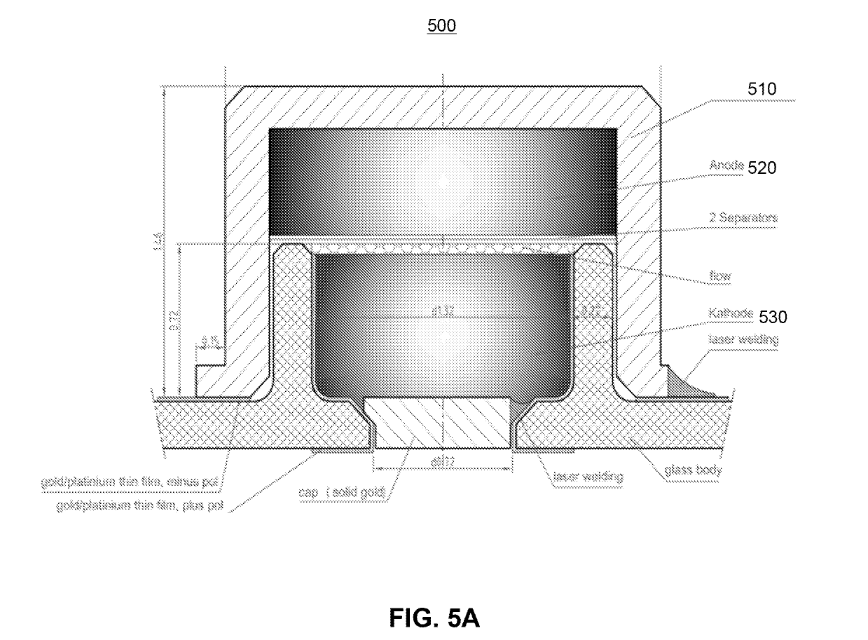

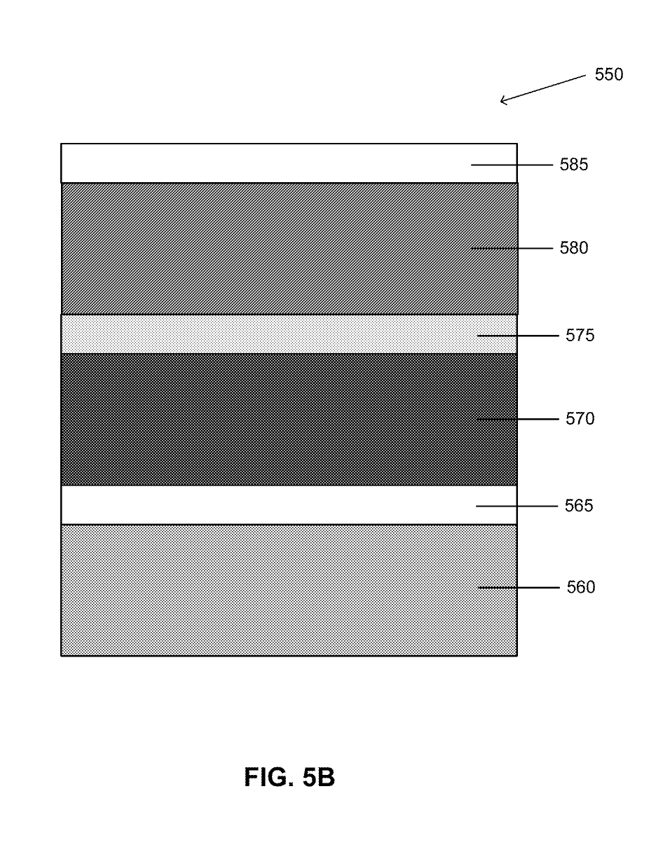

FIG. 5A shows a lithium ion rechargeable battery 500 suitable for use in an implantable device such as the implantable ophthalmic device 100 of FIGS. 1A and 1B. For example, the battery 500 can correspond to the either of the batteries 140 shown in FIG. 2. The battery 500 includes a casing 510, which can be made from gold or any other suitable material. The battery 500 also includes an anode 520 and a cathode 530, which can correspond to the battery cells 141 of FIG. 2. The anode 520 and cathode 530 are separated by two separators 540. The battery 500 can be sealed onto the wafer 106, for example by gold laser welding. In some implementations, the battery 500 can have a capacity of 160 μAh and a lifetime of more than 6000 charging cycles.

FIG. 5B shows a thin-film, solid-state battery 550 that is also suitable for use in an implantable device such as the implantable ophthalmic device 100 of FIGS. 1A and 1B. The battery 550 can be used as either or both of the batteries 140 shown in FIG. 2. In some implementations, the battery 550 can be used together with the lithium ion rechargeable battery 500 of FIG. 5A. The battery 550 is built on a substrate 560 which can be formed from mica. The substrate 560 can have a thickness of about 25 microns. An electrical contact 565 can be formed atop the substrate 560. In some implementations, the contact 565 is formed from platinum and has a thickness of about 0.5 microns. A cathode layer 570 can be formed on top of the electrical contact 565. In some implementations, the cathode 570 can be made from LiCoO₂ and can have a thickness of about 30 microns. An electrolyte layer 575 can be in contact with the cathode 570. In some implementations, the electrolyte layer 575 can be made from LiPON and can have a thickness of about three microns. The electrolyte layer 575 separates the cathode 570 from an anode 580. The anode 580 can be made from lithium and can have a thickness in the range of about 18 microns. A second contact layer 585 can be formed on top of the anode 580. The second contact can be formed from platinum, for example, and can be about 0.5 microns thick.

In some implementations, the entire battery 550 can have a thickness of about 80 microns and an electrical storage capacity of about 11 μAh/mm³. Because the battery 550 is so thin, it can be flexible enough to bend, e.g., for implantation through a small incision in the body. As understood by those of ordinary skill in the art, smaller incisions tend to heal more rapidly and usually accompanied by less swelling than large incisions. As a result, implantations performed with smaller incisions tend to be associated with shorter recovery times, lower complication rates, and less discomfort.

In addition, it may also be safer than other batteries for implantable devices. For example, the battery 550 can be implanted into the eye of a patient as part of an IOL. Because the battery 550 is a solid-state device, there is little to no risk of out gassing or liquid leaks, which means that there is lower risk of eye damage due to a defective or damaged battery.

Battery Charging Circuitry and Processes for Implantable Ophthalmic Devices

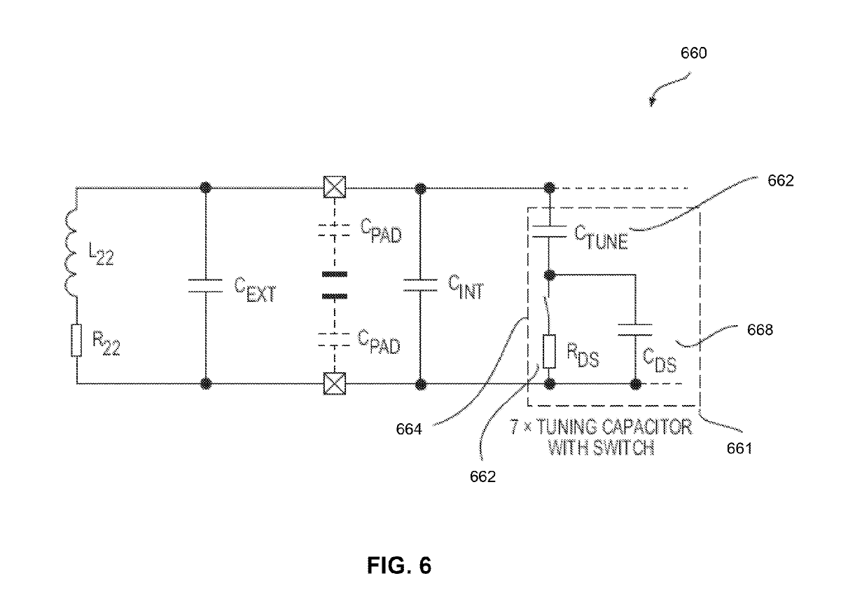

FIG. 6 is circuit diagram showing a battery charging circuit 660 suitable for charging batteries use in an implantable device such as the implantable ophthalmic device 100 shown in FIGS. 1A, 1B, and 2. The battery charger 660 may draw power inductively via an RF antenna, which supplies current to the batteries, for example, via a rectification circuit. The RF antenna can correspond to the RF antenna 190 of FIG. 1A. The battery charging circuit 660 includes one or more trimming blocks 661, each of which includes a tuning capacitor 662 coupled in series with both a switch 664 and a load capacitor 668. As shown in FIG. 6, the switch 664 and load capacitor 668 are in parallel. Closing the switch 664 connects the tuning capacitor 662 to a load 666, increasing the impedance to provide better power flow from an external power supply to the rectification circuit. The trimming blocks 661 can be activated or de-activated as desired to optimize power flow. Once the battery charging circuit 660 is set appropriately, a magnetic field induces current flow in the device. The rectification circuit can then harvest a DC voltage for charging the batteries.

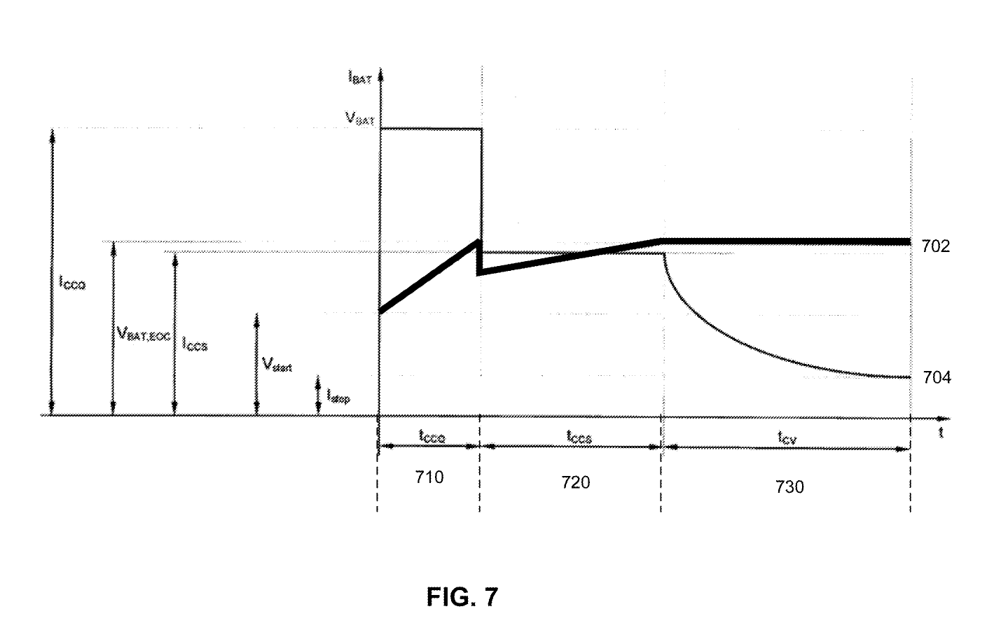

FIG. 7 is a graph that illustrates a charging process (recharging profile) for a rechargeable battery such as the lithium ion battery 500 of FIG. 5A or the solid-state battery 550 of FIG. 5B using the charging circuitry 660 in FIG. 6. The graph shows a battery voltage level 702 and a battery current level 704 over three time intervals: a first time interval 710 (t_{CCQ}), a second time interval 720 (t_{CCS}), and a third time interval 730 (t_{CV}). Applying a larger current during the first time interval 710 and a smaller current during the second time interval 720 can result in a faster overall charging process for the battery.

In some implementations, the current 704 can be applied to the battery by a control module such as the battery charge and power management module 204 of FIG. 2. As shown in the graph, the battery voltage begins from Vₛₜₐᵣₜ at time zero (i.e., the beginning of the first time interval 710). A constant current denoted by I_{CCQ} is applied to the battery throughout the first time interval 710, causing the voltage 702 of the battery to increase linearly until it reaches a predetermined battery charge termination voltage labeled V_{BAT,EOC} at the end of the first time interval 710. The battery charge termination voltage may be a function of the battery and can be detected, for example, by the battery charge and power management module 204 of FIG. 2. The battery charge and power management module 204 determines that the time interval 710 has ended upon sensing the that the battery voltage has reached the battery charge termination voltage.

The current 704 is then reduced to the level denoted by I_{CCS} at the beginning of the second time interval 720, and is held constant throughout the second time interval 720. In some cases, this second current level I_{CCS} is about half the constant current level I_{CCQ} applied during the first time interval 710. The reduction in current causes the battery voltage 702 to drop at the beginning of the time interval 720, but the voltage increases linearly throughout the time interval 720 until it again reaches the charge termination voltage. In some implementations, the rate of increase of the battery voltage 702 is proportional to the level of applied current 704. Thus, during the second time interval 720, the voltage 702 increases at a slower rate due to the decreased current 704.

Upon sensing that the battery voltage 702 has reached the charge termination voltage, the battery control module determines that the second time interval 720 has ended. In response, the battery control module causes the current 704 to decreased until it approaches a level denoted as Iₛₜₒₚ at the end of the time interval 730. The battery control module maintains the battery voltage 702 at the charge termination voltage throughout the third time interval 730 by changing the applied current.

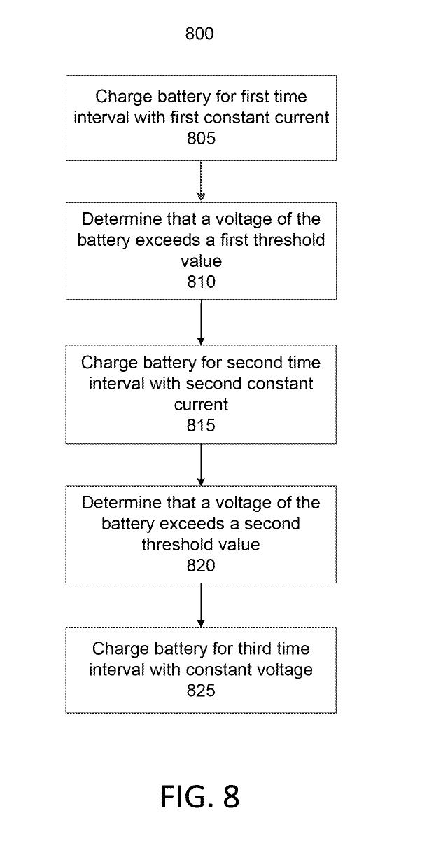

FIG. 8 is a flow diagram of a process 800 for charging a rechargeable battery, according to an illustrative implementation. The process 800 includes charging the battery for a first time interval with a first constant current (step 805). In some implementations, the first time interval can correspond to the time interval 710 of FIG. 7, and the first constant current can correspond to the current level I_{CCQ}. During the first time interval, the battery voltage can increase linearly in response to the first constant current applied. The process 800 can also include determining that a voltage of the battery exceeds a first threshold value (step 810). For example, the first threshold value can be equal to the charge termination voltage V_{BAT,EOC} as shown in FIG. 7. The first time interval can end when the battery voltage meets or exceeds the first threshold value.

The process 800 can include charging the battery for a second time interval with a second constant current (Step 815). In some implementations, the second time interval can correspond to the time interval 720 of FIG. 7. The second current can be less than the first constant current. For instance, the second current may be about 40-60% of the first current (e.g., 45%, 50%, 55%, or any other percentage from 40% to 60%). In some implementations, the second constant current can correspond to the current level I_{CCS}. During the second time interval, the battery voltage can increase linearly in response to the second constant current applied. The rate of increase in voltage can be greater than or less than the rate of increase in voltage experienced by the battery during the first time interval.

The process 800 can also include determining that a voltage of the battery exceeds a second threshold value (step 820) with a control module in an ASIC. In some implementations, the second threshold value can be equal to the first threshold value. For example, the second threshold value can be equal to the charge termination voltage V_{BAT,EOC} as shown in FIG. 7. The second time interval can end when the battery voltage meets or exceeds the second threshold value.

The process 800 can also include charging the battery for a third time interval with a constant voltage (Step 825). For example, the third time interval can correspond to the time interval 730 shown in FIG. 7, and the constant voltage can be equal to V_{BAT,EOC}. In order to achieve the constant voltage, the current applied to the battery can be decreased, e.g., so as to maintain the constant voltage level. As the charge stored by the battery increases, less current is required to maintain the constant voltage. In some implementations, the current applied to the battery can be decreased exponentially, as shown in FIG. 7.

TABLE 1
Battery Management Parameters

| Symbol | Parameter | min | nom | max | Unit |
|---|---|---|---|---|---|
| Vₛₜₐᵣₜ | Battery charge start voltage @ 70% DOD | — | 3.500 | — | mV |
| I_{CCS} | Battery charge current for standard charging | 20 | 25 | 30 | μA |
| | (per battery; regular use) | | | | |
| I_{CCQ} | Battery charge current for quick charging | 40 | 50 | 60 | μA |
| | (per battery; exceptional use for clinical trials) | | | | |
| t_{CCQ} | Constant current mode battery charge time for | — | 1 | 1.2 | hr |
| | quick charging (timeout controlled) | | | | |
| t_{CCS} | Constant current mode battery charge time for | — | 5 | 6 | hr |
| | standard charging (timeout controlled) | | | | |
| t_{CV} | Time for constant voltage mode; used to settle the | — | 2 | 2.4 | hr |
| | charge in the battery (timeout controlled) | | | | |
| Iₛₜₒₚ | Residual battery charge current when CV charge | | | | μA |
| | mode terminates. | | | | |
| V_{BAT,EOC} | Battery charge termination voltage | 4100 | 4150 | 4200 | mV |
| V_{SD} | Battery shutdown voltage | 3450 | 3500 | 3550 | mV |
| V_{BAT,EOD} | Allowed battery discharge voltage before | 2500 | — | — | mV |
| | permanent damage | | | | |

TABLE 1 lists exemplary charging times, currents, and voltages for use in the charging process illustrated in FIGS. 7 and 8. As understood by those skilled in the art, the exact choice of currents and times for defining the recharging profile depends on the battery technology and the recharge strategy. For example, some batteries can be charged at constant voltage. The exact recharging time, recharging percentage of the maximum battery capacity, and battery life may be adjusted to achieve the desired performance. In some cases, the battery charge start voltage Vₛₜₐᵣₜ may be raised or lowered depending on the battery's state of charge (SOC) or depth of discharge (DOD). (The SOC represents a battery's available charge; the DOD represents the amount charge expended by the battery.)

Dual-Battery Discharging

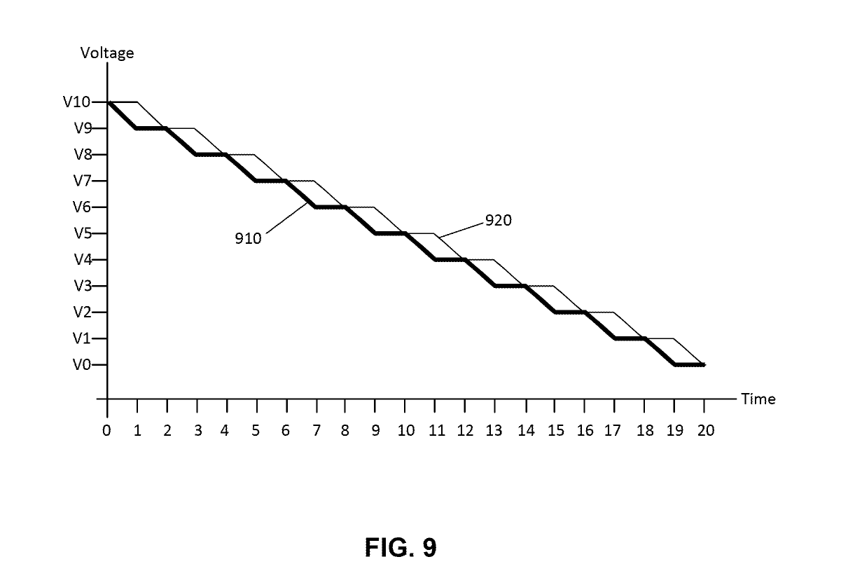

FIG. 9 is a graph showing the discharge of the two batteries 140 as controlled by the battery charge and power management module 204 shown in FIG. 2. The voltage of the first battery is shown by a first line 910 while the voltage of the second battery is shown by the line 920. As depicted in the graph, the voltage of both batteries begins at a voltage denoted by V10 at time 0. In some implementations, the voltage V10 can correspond to a charge termination voltage, such as about 4200 mV. The exact charge termination voltage may depend on the battery technology and the battery's depth of discharge. The charge termination voltage can be measured using a reference voltage, e.g., as provided by the BGR circuit 218 in the low-voltage ASIC 130b (FIG. 2).

In operation, the power management module 204 discharges the first battery linearly over a first time interval, while the second battery remains at a constant voltage. For example, the first battery can be discharged to actuate an electroactive element as discussed above in connection with FIGS. 1A and 1B, while the second battery remains unused. Upon sensing that the voltage of the first battery has decreased by a first increment to a first predetermined lower voltage level (V9 at time 1), the power management module stops discharging the first battery and starts discharging the second battery while the first battery remains at a constant voltage.

In response to sensing that the second battery has reached the first predetermined lower voltage level V9, the power management module stops discharging the second battery and starts discharging the first battery to a second predetermined voltage level V8 while the second battery remains at a constant voltage (V9). The power management module repeats this process iteratively through a series of predetermined voltage levels (V10 through V0) so that the two batteries are discharged substantially simultaneously. In some examples, these predetermined voltage levels are spaced evenly, e.g., at increments of 100 mV. In operation, it may take hours to days for the first and second batteries to reach the ultimate discharge level V0.

The discharging scheme shown in FIG. 9 can help to increase the useful life of an implantable device. For example, the device can be designed such that one battery is sufficient to power the device. Alternately discharging two batteries according to the process shown in FIG. 9 can therefore extend the time between charging cycles. Less frequent charging cycles can also lead to increased battery life over time. Using two or more batteries also mitigates the risk of battery failure; if one battery fails, the other may continue to operate, prolonging the implantable device's useful life.

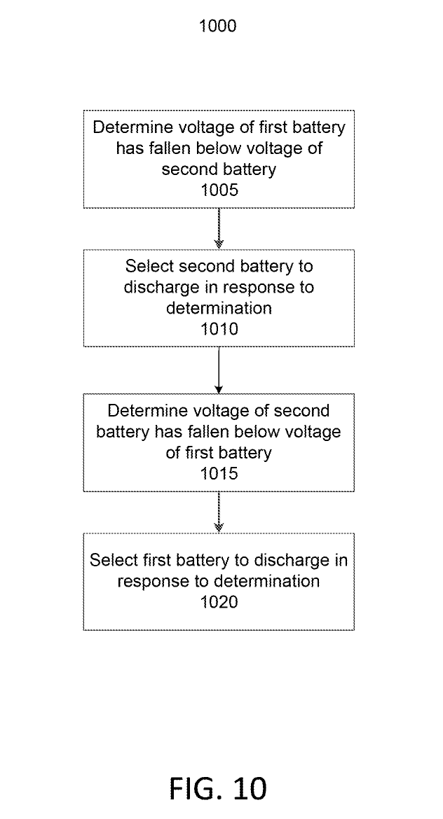

FIG. 10 is a flow diagram of a process 1000 for discharging two rechargeable batteries substantially simultaneously, according to an illustrative implementation. The process 1000 includes determining, with a power management module (e.g., module 204 in FIG. 2) that a voltage of a first battery has fallen below a voltage of a second battery (Step 1005). For example, a voltage sensing circuit and a comparator can be used to determine the relative voltage levels of the first and second batteries. The process 1000 can also include selecting the second battery to discharge in response to determining that the voltage level of the first battery has fallen below the voltage level of the second battery (Step 1010). Electrical power extracted from the second battery as it is discharged can be used to actuate an electroactive element, such as the DOE 260 shown in FIG. 2. While the second battery is discharged (Step 1010), the first battery can be held at a constant voltage, for example by electrically isolating it from the DOE.

The process 1000 includes determining that the voltage of the second battery has fallen below the voltage of the first battery (Step 1015). Because the first battery is held at a constant voltage while the second battery is discharged in Step 1010, the voltage of the second battery will eventually reach a level below the voltage of the first battery. Voltage levels of both batteries can be continuously or periodically monitored and compared in order to make the determination. The process 1000 can also include selecting the first battery to discharge in response to determining that the voltage level of the second battery has fallen below the voltage level of the first battery (Step 1020). The first battery can be discharged to power the DOE that was previously powered by the second battery, while the second battery can be turned off so that it maintains a substantially constant voltage.

In some implementations, the steps of the process 1000 can be performed iteratively. In this way, the first and second batteries can be discharged substantially simultaneously, although only one battery is discharged at any given time. The process 1000 can help to extend the life of a device in which the first and second batteries are used. Because each battery is used for only about half the time that the device is powered on, charging cycles for the batteries are required less frequently and the expected life of the device is increased.

### CONCLUSION

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations.

However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.).

It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. An implantable device comprising:
a first rechargeable battery; and
a processor operably coupled to the first rechargeable battery and configured to:
charge the first rechargeable battery for a first time interval using a first constant current;
charge the first rechargeable battery for a second time interval using a second constant current less than the first constant current; and
charge the first rechargeable battery for a third time interval using a constant voltage.

2. The implantable device of claim 1, wherein the first rechargeable battery comprises at least one of a solid-state lithium battery and a lithium-ion battery.

3. The implantable device of claim 1, wherein the first rechargeable battery has a volume of less than five cubic millimeters.

4. The implantable device of claim 1, wherein the processor is further configured to determine an end of the first time interval when a voltage of the first rechargeable battery exceeds a first threshold voltage.

5. The implantable device of claim 4, wherein the processor is further configured to determine an end of the second time interval when the voltage of the first rechargeable battery exceeds a second threshold voltage.

6. The implantable device of claim 1, wherein the second constant current is substantially equal to half the first constant current.

7. The implantable device of claim 6, wherein the first constant current is from about 40 μA to about 60 μA.

8. The implantable device of claim 1, wherein the processor further comprises:
a power conversion module configured to (i) receive power from a power source external to the implantable device and (ii) convert the power to the first constant current, the second constant current, and the constant voltage.

9. The implantable device of claim 8, wherein the power source comprises at least one of a radio-frequency source and a light source.

10. The implantable device of claim 1, further comprising:
a second rechargeable battery operably coupled to the processor, wherein the processor is further configured to:
charge the second rechargeable battery for a fourth time interval using a third constant current;
charge the second rechargeable battery for a fifth time interval using a fourth constant current less than the third constant current; and
charge the second rechargeable battery for a sixth time interval using a second constant voltage.

11. The implantable device of claim 1, further comprising:
an electro-active element operably coupled to the processor and configured to modulate at least one optical characteristic of the implantable device.

12. A method of charging a battery, the method comprising:
charging the rechargeable battery for a first time interval using a first constant current;
determining that a voltage of the rechargeable battery exceeds a first threshold value;
charging the rechargeable battery for a second time interval using a second constant current less than the first constant current;
determining that the voltage of the rechargeable battery exceeds a second threshold value; and
charging the rechargeable battery for a third time interval using a constant voltage.

13. An intraocular optic comprising:
an electro-active element configured to vary at least one optical characteristic of the intraocular optic;
a sensor configured to generate a sensor signal within less than about 100 milliseconds in response to sensing at least one of a change in light level and a physiological response;
a first control circuit, operably coupled to the sensor, configured to sample the sensor signal and to generate an actuation signal within 100 milliseconds of sampling the sensor signal in response to the sensor signal;
a second control circuit, operably coupled to the first control circuit and to the electro-active element, configured to:
(i) receive the actuation signal,
(ii) transition from a low-power state to a high-power state and actuate the electro-active element within about 5 milliseconds of receiving the actuation signal so as to vary the at least one optical characteristic of the intraocular optic in response to the actuation signal, and
(iii) transition from the high-power state to the low-power state within about 5 milliseconds of actuating the electro-active element so as to minimize current leakage from the second control circuit.

14. The intraocular optic of claim 13, wherein the first control circuit is configured to sample the sensor signal at a period of about 200 milliseconds to about 310 milliseconds.

15. The intraocular optic of claim 13, wherein the first control circuit is configured to sample the sensor signal aperiodically.

16. A method of altering at least one optical characteristic of an intraocular optic in response to at least one of a change in light level and a physiological response, the method comprising:
(A) sensing the at least one of the change in light level and the physiological response;
(B) generating a sensor signal within about 100 milliseconds of sensing the at least one of the change in light level and a physiological response;
(C) sampling the sensor signal with a first control circuit;
(D) generating an actuation signal, with the first control circuit, within 100 milliseconds of sampling the sensor signal;
(E) actuating the intraocular optic based on the actuation signal so as to minimize current leakage from the second control circuit;
(F) receiving the actuation signal at a second control circuit;
(G) transitioning the second control circuit from a low-power state to a high-power state in response to the actuation signal;
(H) actuating an electro-active element with the second control circuit so as alter the at least one characteristic of the intraocular optic within about 5 milliseconds of receiving the actuation signal; and
(I) transitioning the second control circuit from the high-power state to the low-power state within about 5 milliseconds of actuating the electro-active element so as to minimize current leakage from the second control circuit.

17. An implantable device comprising:
a first rechargeable battery having a first voltage;
a second rechargeable battery having a second voltage; and
a processor, operably coupled to the first rechargeable battery and the second rechargeable battery, configured to iteratively:
determine that the first voltage has fallen below the second voltage;
select the second rechargeable battery to discharge in response to the determination that the first voltage has fallen below the second voltage;
determine that the second voltage has fallen below the first voltage; and
select the first rechargeable battery to discharge in response to the determination that the first voltage has fallen below the second voltage.

18. The implantable device of claim 18, wherein at least one of the first rechargeable battery and the second rechargeable battery comprises at least one of a solid-state lithium battery and a lithium-ion battery.

19. The implantable device of claim 18, wherein at least one of the first rechargeable battery and the second rechargeable battery has a volume of less than five cubic millimeters.

20. The implantable device of claim 18, wherein the processor is further configured to:
determine that the first voltage has fallen below a first threshold;
determine that the second voltage had fallen below a second threshold; and
cause a reduction in power flow from the first rechargeable battery and the second rechargeable battery in response to the determination that the first voltage has fallen below the first threshold and the determination that the second voltage had fallen below the second threshold.

21. The implantable device of claim 18, further comprising:
an electro-active element operably coupled to the processor, the first rechargeable battery, and the second rechargeable battery and configured to vary at least one optical characteristic of the implantable device when powered by at least one of the first rechargeable battery and the second rechargeable battery.

22. An intraocular implant comprising:
a sensor configured to sense at least one of a light level and a physiological response;
an electro-active element to vary at least one optical characteristic of the intraocular implant;
a first control circuit, operably coupled to the sensor, configured to sample the sensor signal and to generate an actuation signal within 100 milliseconds of sampling the sensor signal in response to the sensor signal;
a second control circuit, operably coupled to the first control circuit and to the electro-active element, configured to:
(i) receive the actuation signal,
(ii) transition from a low-power state to a high-power state and actuate the electro-active element so as to vary the at least one optical characteristic of the intraocular optic in response to the actuation signal, and
(iii) transition from the high-power state to the low-power state of actuating the electro-active element so as to minimize current leakage from the second control circuit; and
at least one rechargeable battery, operably coupled to the first control circuit and the second control circuit, configured to provide power to the second control circuit when the second control circuit is in the high-power state and to be recharged by:
(i) a first constant current provided by the first control circuit over a first time interval,
(ii) a second constant current less than the first constant current provided by the first control circuit over a second time interval after the first time interval, and
(iii) a constant voltage provided by the first control circuit over a third time interval after the second time interval.

23. The intraocular implant of claim 22, wherein the at least one rechargeable battery comprises a first rechargeable battery having a first voltage and a second rechargeable battery having a second voltage and wherein the first control circuit is further configured to provide power to the second control circuit by iteratively:
determining that the first voltage has fallen below the second voltage;
selecting the second rechargeable battery to discharge in response to the determination that the first voltage has fallen below the second voltage;
determining that the second voltage has fallen below the first voltage; and
selecting the first rechargeable battery to discharge in response to the determination that the first voltage has fallen below the second voltage.
